(19) United States
(12) Patent Application Publication
(10) Pub. No.: US 2014/0328944 A1
(43) Pub. Date: Nov. 6, 2014

(54) USE OF CTBP1 siRNA FOR THE TREATMENT OF GASTRIC CANCER

(71) Applicants: Manuel Gidekel, Santiago (CL); Carolina Bizama, Santiago (CL); Osvaldo Podhajcer, Washington, DC (US)

(72) Inventors: Manuel Gidekel, Santiago (CL); Carolina Bizama, Santiago (CL); Osvaldo Podhajcer, Washington, DC (US)

(73) Assignee: CTI

(21) Appl. No.: 13/986,437

(22) Filed: May 2, 2013

(51) Int. Cl. C12N 15/113; A61K 33/24; A61K 31/704; A61K 31/7072

(52) U.S. Cl. CPC C12N 15/113; A61K 31/7072; A61K 33/24; A61K 31/704
USPC 424/649; 514/44 R; 514/34

(57) ABSTRACT

This invention encompasses a novel composition comprising silenced CTBP-1 combined with a compound selected from among 5-FU, Cisplatin and Epirubicin. The invention further relates to a method of treating gastric cancer with a combination of siRNA silenced CTBP-1 and a compound selected from among 5-FU, Cisplatin and Epirubicin.

### FIELD OF THE INVENTION

This invention encompasses a novel composition comprising silenced CTBP-1 combined with a compound selected from among 5-FU, Cisplatin and Epirubicin. The invention further relates to a method of treating gastric cancer with a combination of siRNA silenced CTBP-1 and a compound selected from among 5-FU, Cisplatin and Epirubicin.

### BACKGROUND OF THE INVENTION

Worldwide, gastric cancer is considered the fourth most common cancer and second most deadly cancer (Brenner et al., 2009). The survival of resected gastric cancer is estimated between 10-30% (Dicken et al., 2005). Using adjuvant therapy, either chemotherapy or radiotherapy to improve the survival in advanced lesions after surgery.

In spite of multiple schemes, only 30% of patients presented a clinical response, the main complications being recurrence and systemic involvement (Catalano et al., 2005).

The most common chemotherapy protocol for gastric cancer is based on the use of three drugs in conjunction 5-Fluorouracil (5-FU), Cisplatin and Epirubicin.

5-FU is known as an anti-metabolite that acts as an inhibitor of thymidylate synthase; Epirubicin as an inhibitor of topoisimerasa II; Cisplatin is a DNA intercalating compound that leads damage to DNA in cancer cells. However, the limited benefit and high toxicity of this scheme makes necessary the continued search for new molecular therapeutic approaches designed to reverse or overcome resistance to drugs such as the use of siRNA.

The gene CTBP1 encodes for a phosphoprotein that acts in the nucleus as a transcriptional repressor and in the cytoplasm in the maintenance of vesicular membranes (Birts et al., 2010). Early studies indicate the role of CTBPs in regulation of cellular transformation: E1A loses its C-terminal through interaction with CTBP domain, which makes E1A it less effective in promoting cell transformation, when collaborating with the mutant RAS. Recently, control of the deregulation of the abundance of CTBP1 has been identified as a key step in the initiation of formation of colon tumors: CTBP1 degradation depends on APC in these cells and APC mutation leads to increased CTBP1 resulting in the initiation of the formation of adenomas (Nadauld et al., 2006) Apart from their roles in the initiation and progression of tumorigenesis, CTBPs have also been implicated in the cellular response to chemotherapy.

### SUMMARY OF THE INVENTION

The combination of existing chemotherapy with the introduction of intra-tumoral siRNA CTBP1 provides a promising therapeutic strategy for patients with gastric cancer resistant to chemotherapeutic agents.

In one embodiment of the present invention, a method of treating gastric cancer is provided comprising the steps of:
(1) silencing gene CTBP-1 with siRNA;
(2) preparing a combination of the silenced CTBP-1 gene with an anti-cancer compound selected from the group consisting of 5-FU, Cisplatin and Epirubicin or mixtures. thereof;
(3) administering a therapeutic dosage of the combination to a patient with gastric cancer.

In another embodiment of the present invention, a composition for treating gastric cancer is provided comprising CTBP-1 gene silenced with siRNA combined with an anti-cancer compound selected from the group consisting of 5-FU, Cisplatin and Epirubicin or mixtures thereof. Preferably the anti-cancer compound is 5-FU wherein the activity of the 5-FU against expressed cancer cell lines is enhanced by a factor of approximately 6; when the anticancer compound is Cisplatin the activity of Cisplatin against expressed cancer cell lines is enhanced by a factor of 4-5; and, when the anticancer compound is Epirubicin the activity of Epirubicin against expressed cancer cell lines is enhanced by a factor of 12-17.

Small interfering RNA (si RNA) are small pieces of double-stranded RNA, usually about 21 nucleotides long, with nucleotide overhangs at each end that can be used to interfere with the translation of protein by binding to and promoting the degradation of messenger RNA at specific sequences. Therefore they may prevent the production of specific proteins based on the nucleotide sequence of their corresponding mRNA. The process is called RNA interference and may also be referred to as si RNA silencing

### DETAILED DESCRIPTION OF THE INVENTION

### Materials and Methods

### Cell Lines and Drugs

MKN45 cell lines were grown in RPM1-1640 media and AGS cell lines in F12K, both media were supplemented with 10% characterized fetal bovine serum (FBS) (Hyclone, Logan, Utah, USA) and 100 U /ml of Penicillin /Streptomycin (Gibco BRL, Gaithersburgh, Md., USA). Cells were grown in an incubator (NUAIRE, Plymouth, USA) at 37° C. with an atmosphere of 5% CO2 and 90% humidity. The drugs 5-FU, Cisplatin and Epirubicin were donated for this research by the Kampar Oncology Laboratory (Santiago, Chile).

### Transfection with siRNA

In 6-well plates, 200,000 cells were cultured in their respective culture medium with 10% FBS for 24 h at 37° C. and 5% CO2 pressure. Then cells were washed once with PBS IX and 900 ul of their respective medium without serum and without antibiotic were added.

The cells were transfected with a pool of 4 siRNA Flexitube siRNA CTBP1 and siRNA control AllStars Negative Control (QIAGEN, Valencia, Calif., USA) at a concentration of 5 nM with 2.5 ul of Lipofectamine 2000 (Invitrogen, Carlsbad, Calif., USA). After 7 h of incubation, the transfection medium was removed and replaced by the respective culture medium supplemented with 10% FBS (Hyclone, Logan, Utah, USA) and 100 U/mL of Penicillin plus 100 ug/mL of Streptomycin (Gibco BRL, Gaithersburgh, Md., USA).

### Analysis of Cell Proliferation

24 hours after transfection, cells were seeded in 96 well plates at a concentration of 10,000 cells per well. After 24 hours, cells were faced with serial dilutions of each chemotherapeutic agent. The cell proliferation assay was performed after waiting 72 hours of incubation with each drug and it was performed using MTS-based cell proliferation assay (CellTitre Aqueous One Cell Proliferation assay; Promega). Each experiment was performed in triplicate. The IC50 values were calculated using GraphPad 5.0 software. The results of the response to the drugs were expressed as mean±SD.

The gene CTBP1 encodes for a phosphoprotein that acts in the nucleus as a transcriptional repressor and in the cytoplasm in the maintenance of vesicular membranes (Birts et al., 2010). Early studies indicate the role of CTBPs in regulation of cellular transformation: E1A loses its C-terminal through interaction with CTBP domain, which makes E1A it less effective in promoting cell transformation, when collaborating with the mutant RAS. Recently, control of the deregulation of the abundance of CTBP1 has been identified as a key step in the initiation of formation of colon tumors: CTBP1 degradation depends on APC in these cells and APC mutation leads to increased CTBP1 resulting in the initiation of the formation of adenomas (Nadauld et al., 2006) Apart from their roles in the initiation and progression of tumorigenesis, CTBPs have also been implicated in the cellular response to chemotherapy.

Previous studies have shown that reducing CTBP1 increases sensitivity to 5-FU in breast cancer (Birts et al., 2010). It is believed that the role of CTBP1 in chemotherapy could be due to the affect of the protein in signaling pathways that affect cell survival and cell proliferation, such as genetic suppression of proapoptotic genes and the maintenance of mitotic fidelity (Bergman et al., 2009).

Additionally, it has been demonstrated that this gene could promote drugs resistance by increased expression of the MDR1 gene (multi-drug resistance 1) (Jin et al., 2007).

From previous experiments conducted by the present inventors using PCR Pathway array methodology of the Wnt signaling pathway, it was discovered that deregulation of the Wnt pathway is mainly given by the activation of two ways: in the non-canonical/JNK pathway and the calcium-mediated signaling pathway. Standing out in the canonical pathway Wnt gene over-expression CTBP1 known as an antagonist of this pathway. The level of expression of this gene in tumor tissues was 70.8 times higher than the controls of normal tissue adjacent to tumor.

This gene is also expressed in gastric cancer lines SNU1, SNU16, Kato III, MKN45, AGS and N87 (FIG. 1). In carrying out the silencing of this gene in the cell line AGS using different concentrations of siRNA, it was observed that this gene expression decreases between 67-70% at 48 hrs post-transfection (FIG. 2). Subsequently, it was assessed whether the silencing of this gene affects the sensitivity to the treatment of three drugs commonly used in chemotherapy: 5-FU, Cisplatin and Epirubicin. As shown in FIG. 3, in cell lines AGS and MKN45, silencing of CTBP1 with siRNA increases approximately 6 times the sensitivity to 5-FU, 4 to 5 times the sensitivity to Cisplatin and 12-17 times the sensitivity to Epirubicin in relation to untreated cells (WT) and the ones transfected with the control siRNA. With these results we demonstrate that treatment with siRNA enhances the activity of 5-FU, Cisplatin and Epirubicin in the dose-response curves for these drugs.

With these results it is postulated that inhibition of CTBP1 is a new therapeutic target of interest for the treatment of gastric cancer, because it can enhance the effect of chemotherapeutic agents often used in the clinic.

The combination referred to herein relates to either a physical mixture of silenced CTBP-1 with the known anti-cancer compound or to separate administration of each component of the combination.

### BRIEF DESCRIPTION OF THE FIGURES

FIG. 1. CTBP1 expression in gastric cancer cell lines.

FIG. 2. Validation by PCR in real silencing time of CTBP1 at different siRNA concentrations, 48 hrs post-transfection with siRNA.

FIG. 3. CTBP1 silencing increases sensitivity to 5-FU, Cisplatin and Epirubicin in two gastric cancer cell lines. The figures show the mean±standard deviation of the result of three separate experiments.

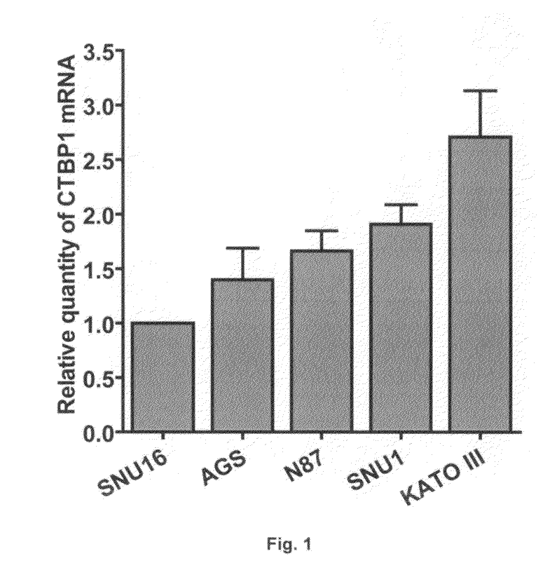

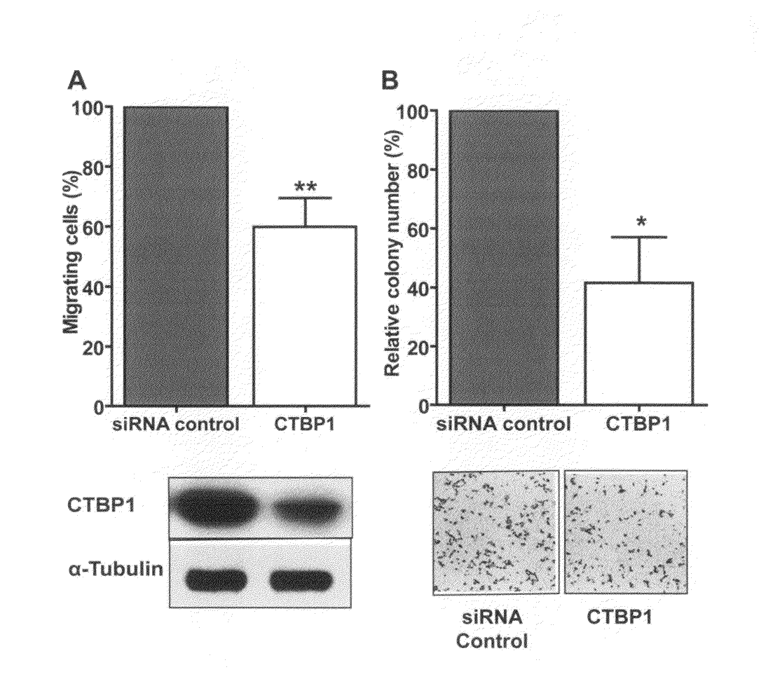

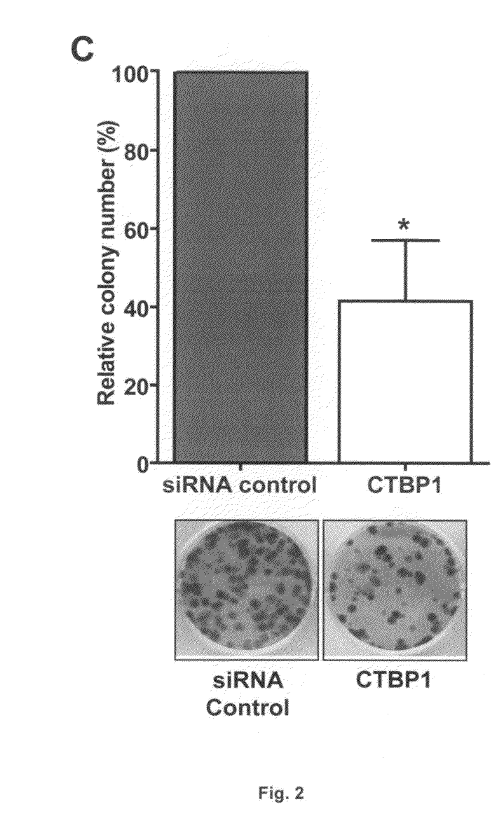

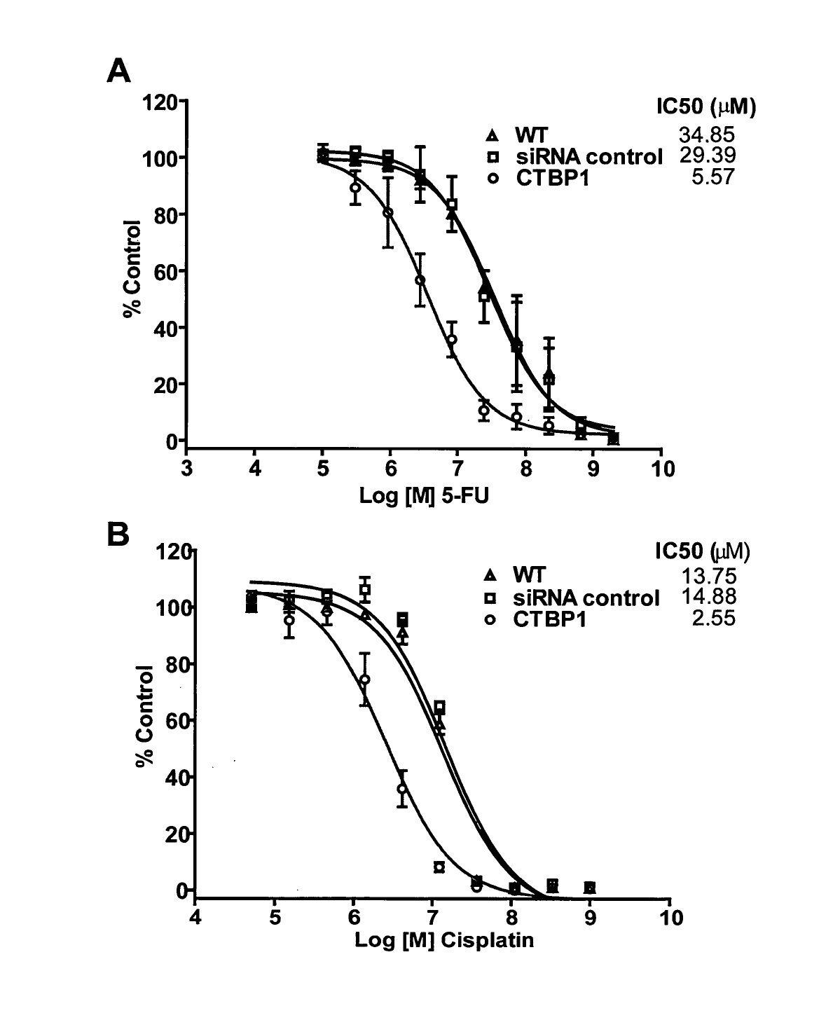

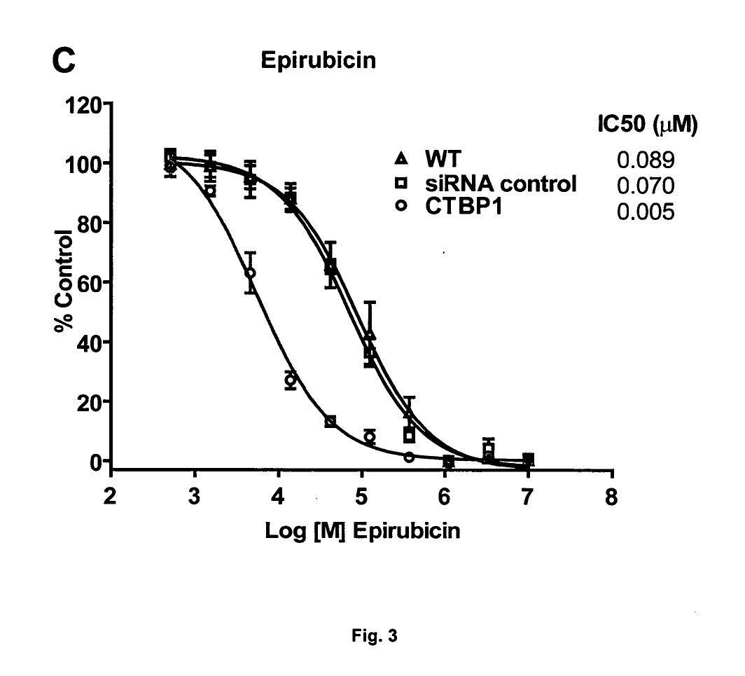

### REFERENCES

Bergman, L. M., Birts, C. N., Darley, M., Gabrielli, B., and Blaydes, J. P. (2009). Promote cell survival CtBPs Through the maintenance of mitotic fidelity. Mol Cell Biol 29, 4539-4551.

Birts, C. N., Harding, R., Soosaipillai, G., Haider, T., Azim-Araghi, A., Darley, M., Cutress, R. I., Bateman, A. C., and Blaydes, J. P. (2010). Expression of CtBP family protein isoforms in breast cancer and their role in chemoresistance. Cell Biol 103, 1-19.

Brenner, H., Rothenbacher, D., and Arndt, V. (2009). Epidemiology of stomach cancer. Methods Mol Biol 472, 467-477.

Catalano, V., LaBianca, R., Beretta, G. D., Gatta, G., Braud, F., and Van Cutsem, E. (2005). Gastric cancer. Crit Rev Hematol Oncol 54, 209-241.

Dicken, B. J., Bigam, D. L., Cass, C., Mackey, J. R., Joy, A. A., and Hamilton, S. M. (2005). Gastric adenocarcinoma: review and future Considerations for directions. Ann Surg 241, 27-39.

Jin, W., Scotto, K. W., Hait, W. N., and Yang, J. M. (2007). Involvement of CtBP1 in the transcriptional activation of the MDR1 gene in human multidrug resistant cancer cells. Biochem Pharmacol 74, 851-859.

Nadauld, L. D., Phelps, R., Moore, B. C., Eisinger, A., Sandoval, I. T., Chidester, S., Peterson, P. W., Manos, E. J., Sklow, B., Burt, R. W., and Jones, D. A. (2006). Control of adenomatous polyposis coli C-terminal binding protein-1 Stability Regulates expression of intestinal retinol dehydrogenases. J Biol Chem 281,

[symbol]

1. A method of treating gastric cancer comprising the steps of:
(1) silencing gene CTBP-1 with siRNA;
(2) preparing a combination of the silenced CTBP-1 gene with an anti-cancer compound selected from the group consisting of 5-FU, Cisplatin, Epirubicin and mixtures thereof;
(3) administering a therapeutic dosage of the combination to a patient with gastric cancer either separately or physically mixed.

2. A composition for treating gastric cancer comprising CTBP-1 gene silenced with siRNA combined with an anti-cancer compound selected from the group consisting of 5-FU, Cisplatin and Epirubicin or mixtures thereof.

3. A composition according to claim 2 wherein the anti-cancer compound is 5-FU and wherein the activity of 5-FU against expressed cancer cell lines is enhanced by a factor of approximately 6.

4. A composition according to claim 2 wherein the anticancer compound is Cisplatin wherein the activity of Cisplatin against expressed cancer cell lines is enhanced by a factor of 4-5.

5. A composition according to claim 2 wherein the anticancer compound is Epirubicin wherein the activity of Epirubicin against expressed cancer cell lines is enhanced by a factor of 12-17.
